(19) United States
(12) Patent Application Publication
(10) Pub. No.: US 2026/0294319 A1
(43) Pub. Date: Oct. 1, 2026

(54) SYSTEMS AND METHODS FOR ANALYZING RISK OF CARDIAC ARREST

(71) Applicant: CEDARS-SINAI MEDICAL CENTER, Los Angeles, CA (US)

(72) Inventors: Sumeet S. CHUGH, Santa Monica, CA (US); David OUYANG, Los Angeles, CA (US)

(73) Assignee: CEDARS-SINAI MEDICAL CENTER, Los Angeles, CA (US)

(21) Appl. No.: 19/477,742

(22) PCT Filed: May 4, 2024
(86) PCT No.: PCT/US2024/027892

§ 371 Date: Oct. 22, 2025

Related U.S. Application Data
(60) Provisional application No. 63/500,550, filed on May 5, 2023.

(51) Int. Cl. A61B 5/361; A61B 5/00; A61B 5/28

(52) U.S. Cl. CPC A61B 5/361; A61B 5/28; A61B 5/7267; A61B 5/7275; A61B 5/742

(57) ABSTRACT

A method for analyzing risk of cardiac arrest in a subject includes receiving electrocardiograph (ECG) data of a subject; inputting at least a portion of the ECG data into a trained machine learning model; and receiving from the trained machine learning model an indication of a risk of cardiac arrest in the subject.

### CROSS-REFERENCE TO RELATED APPLICATIONS

This application claims priority to and the benefit of U.S. Provisional Patent Application. No. 63/500,550, filed May 5, 2023, which is hereby incorporated by reference herein in its entirety.

### STATEMENT REGARDING FEDERALLY SPONSORED RESEARCH

This invention was made with government support under Grant Nos. HL145675 and HL147358 awarded by the National Institutes of Health. The government has certain rights in the invention.

### TECHNICAL FIELD

The present disclosure relates generally to systems and methods for analyzing risk of cardiac arrest, and more particularly, to systems and methods for analyzing electrography data using deep learning algorithms to determine risk of cardiac arrest.

### BACKGROUND

Sudden cardiac arrest is a major public health problem, and most cases occur unexpectedly without prior detection of high cardiac arrest risk. Current methods for analyzing risk of cardiac arrest are generally insufficient. Thus, new systems and methods for analyzing risk of cardiac arrest are needed.

### SUMMARY

According to some implementations of the present disclosure, a method for analyzing risk of cardiac arrest in a subject includes receiving electrocardiograph (ECG) data of a subject; inputting at least a portion of the ECG data into a trained machine learning model; and receiving from the trained machine learning model an indication of a risk of cardiac arrest in the subject.

According to some implementations of the present disclosure, a method of training a machine learning model to determine a risk of cardiac arrest in a subject, the method comprises: receiving electrocardiograph (ECG) data; forming a training dataset from the ECG data, the training dataset including an active portion and a control portion, wherein the active portion includes a first plurality of sets of ECG data, each generated from a patient that experienced a subsequent cardiac arrest, and the control portion includes a second plurality of sets of ECG data, each generated from a control subject that did not experience a subsequent cardiac arrest; and training the machine learning model using the training dataset.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

The disclosure, and its advantages and drawings, will be better understood from the following description of representative embodiments together with reference to the accompanying drawings. These drawings depict only representative embodiments, and are therefore not to be considered as limitations on the scope of the various embodiments or claims.

FIG. 1 is a block diagram of a system for analyzing risk of cardiac arrest in a subject, according to aspects of the present disclosure.

FIG. 2 is a flow chart of a method for, according to aspects of the present disclosure.

FIG. 3 shows the breakdown of the internal and external cohorts for training, testing, and validation a model for analyzing risk of cardiac arrest in a subject, according to aspects of the present disclosure.

FIG. 4 shows the flow of testing, training, and validation the model, according to aspects of the present disclosure.

FIG. 5 shows a first set of ROC curves for analyzing the performance of the model, according to aspects of the present disclosure.

FIG. 6 shows a second set of ROC curves for analyzing the performance of the model, according to aspects of the present disclosure.

### DETAILED DESCRIPTION

Sudden cardiac arrest (SCD, also referred to as sudden cardiac death or SCD) is a major, global public health problem. In Europe and the United States, ~700,000 individuals will suffer from this mostly lethal condition on a yearly basis. Given the high mortality rate of SCA, effective primary prevention could make a substantial positive impact but the current approach needs augmentation. Based on randomized clinical trials, patients identified to be at high risk based on severely reduced left ventricular systolic function (LVEF <35%) receive implantable cardioverter-defibrillators. However, there is no existing risk stratification methodology for individuals with LVEF >35% that make up 70% of community SCA. Moreover, ~40-50% of all SCA cases occur in individuals without previously diagnosed cardiac disease, which is a prerequisite for SCA risk assessment. However, conventional electrocardiogram (ECG)-based risk stratification tools are usually limited by low accuracy or practicality, since they include measurements that are not part of a usual ECG interpretation, thus requiring customized measurement or trained medical personnel interpretation.

The ECG is the most inexpensive and widely available cardiac test and can now also be measured by wearable technology with increasing accuracy. Disclosed herein is an ECG-based deep learning (DL) model to identify individuals at high risk of SCA.

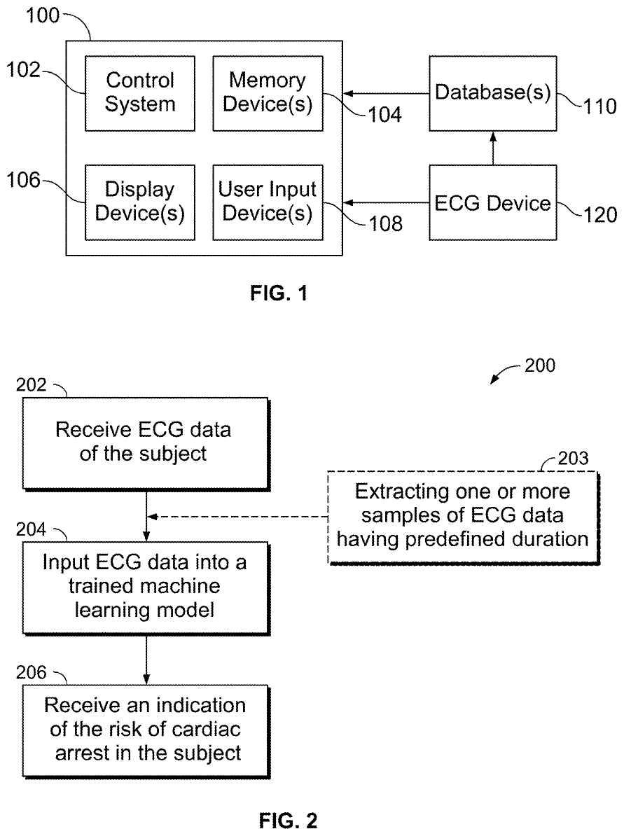

FIG. 1 illustrates a block diagram of system 100 that can be used to analyze risk of cardiac arrest in a subject. The system 100 includes a control system 102, one or more memory devices 104, one or more display devices 106, and one or more user input devices 108. The control system 102 can generally include one or more units, which may include a processing unit of any suitable processing device, including general purpose computer systems, microprocessors, digital signal processors, micro-controllers, application specific integrated circuits (ASICs), programmable logic devices (PLDs) field programmable logic devices (FPLDs), programmable gate arrays (PGAs), field programmable gate arrays (FPGAs), mobile devices such as mobile telephones, personal digital assistants (PDAs), or tablet computers, local servers, remote servers, wearable computers, or the like.

The one or more memory devices 104 can generally include any suitable memory device, including solid-state memories, optical media, magnetic media, random access memory (RAM), read only memory (ROM), a floppy disk, a hard disk, a CD ROM, a DVD ROM, flash memory, any other computer readable medium that is read from and/or written to by a magnetic, optical, or other reading and/or writing system, and the like. The one or more display devices 106 can generally include any suitable display device, such as an LCD display, an LED display, an OLED display, a television, a laptop screen, a touch screen, or the like. The one or more user input devices 108 can generally include any suitable user input device, including a keyboard, a mouse, a microphone (for receiving voice input), a touch screen, and the like.

In some implementations, some elements of the system 100 may be combined into a single device. For example, the control system 102 and the display device 106 may combined into a single device (e.g., a desktop computer or a laptop computer with a screen). In another example, a touchscreen can form both the display device 106 and the user input device 108. The one or more memory devices 104 can store computer-readable instructions that can be executed by the control system 102 to implement one or more methods for analyzing the risk of cardiac arrest in the subject.

In some implementations, the system 100 is communicatively coupled to one or more databases 110. The one or more databases 110 can include any data that is needed by the system 100. For example, the databases 110 can store ECG data used by the control system 102 to determine the risk of cardiac arrest. In another example, the databases 110 may store computer-readable instructions that can be executed by the control system 102 to aid in implementing one or more methods for determining risk of cardiac arrest.

In some implementations, the system 100 is communicatively coupled to an ECG device 120. The ECG device 120 can be any suitable device for obtaining ECG measurements (e.g., for obtaining ECG data), such as a 3-lead ECG device, a 4-lead ECG device, a 12-lead ECG device, a 3-electrode ECG device, a 5-electrode ECG device, a 10-electrode ECG device, a Holter monitor, a smartwatch, etc. ECG data generated by the ECG device 120 can be transmitted to the system 100 (e.g., to the one or more memory devices 104 of the system 100) and/or to the one or more databases 110.

FIG. 2 shows a flow chart of a method 200 for determining the risk of cardiac arrest in a subject, which will generally be a human being. The risk that is determined using method 200 is the risk of what is generally referred to as sudden cardiac arrest (SCA), which is generally defined as the sudden loss of heart activity, often caused by an issue with the heart's electrical system. Sudden cardiac arrest is different from a heart attack (also referred to as a myocardial infarction), which occurs when blood flow in the coronary artery of the heart decreases and/or stops. Method 200 can be implemented by a system (such as system 100) that includes a control system (such as control system 102) and a memory device (such as the one or more memory devices 104).

Step 202 of the method 200 includes receiving ECG data of the subject. The ECG data can generally be any type of ECG data. For example, the ECG data can include ECG data from a 12-lead ECG, a 3-lead ECG, a 4-lead ECG, a 3-electrode ECG, a 5-electrode ECG, a 10-electrode ECG, a Holter monitor, a smartwatch, or any other suitable source of ECG data. In general, the ECG data can include data indicative of the voltage (also referred to as the potential) of a given ECG lead versus time.

Step 204 of the method 200 includes inputting at least a portion of the ECG data into a trained machine learning model. In some implementations, the portion of the ECG data that is input into the model is a sample of ECG data has a predefined duration. For example, the portion of ECG data input into the model may be ECG data indicative of the voltage of one or more of the ECG leads over a certain period of time.

In some implementations, method 200 includes step 203 that occurs between steps 202 and 204. Step 203 includes extracting one or more samples of ECG data having a predefined duration, to thereby form the portion of the ECG data that is input into the trained machine learning model in step 204. In implementations where the ECG data is ECG data from an n-lead ECG, step 203 can include extracting a sample from each of the n leads. For example, if the ECG data is ECG data from a 12-lead ECG data, step 203 can include extracting 12 separate samples of ECG data, which can then be input into the model at step 204. In some implementations, each of the n samples from the n-lead ECG data has the same predefined duration. In other implementations, at least two of the n samples from the n-lead ECG data may have different predefined durations. In any of these implementations, any sample of ECG data (e.g., a sample of data from any ECG lead) can have a predefined duration (e.g., can be indicative of the voltage over a certain period of time0 that is less than or equal to 10 seconds, less than or equal to 5 seconds, less than or equal to 2.5 seconds, or generally equal to or falling within any other suitable period of time.

Step 206 includes receiving an indication of the subject's risk of cardiac arrest from the trained machine learning model. The model can be trained to output any suitable indication of risk, such as the probability that the subject will experience cardiac arrest within a predetermined period of time (e.g., within the next week, within the next month, within the next year, within the next 5 years, within the next 10 years, etc.); an indication of whether or not the subject will experience cardiac arrest within a predetermined period of time (which may be defined as the probability of experiencing cardiac arrest with the predetermined period of time satisfying a certain threshold probability of risk), or any other suitable indicator. This risk indicator can be used in some cases to implement new monitoring/treatment or to update existing monitoring/treatment. For example, if the output of the machine learning model indicates that the patient is at risk of suffering cardiac arrest within the next year (e.g., 50% chance or greater), than a physician may prescribe certain medicine to the patient, or require the patient to come back for more frequent monitoring. Thus, the use of method 200 can result in more effective monitoring and treatment of patients.

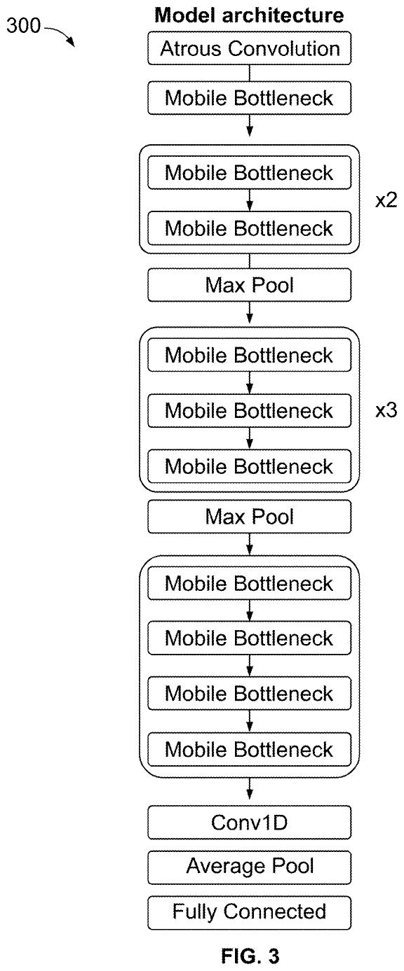

An example implementation of the trained machine learning model is shown in FIG. 3, which illustrates a deep learning (DL) model 300. In this example, the DL model 300 is a convolutional neural network trained to interpret 12-lead ECG data. The number of layers is designed to minimize model complexity and optimize model runtime. The DL model 300 starts with atrous convolutions, which are then followed by a series of convolution layers. Each of these convolution layers has an inverted residual structure, where the input and the output of the convolution layer include bottleneck layers (e.g., 1×1 convolutions), with an intermediate expansion layer (e.g., a 3×3 depth-wise convolution) between the bottleneck layers. To allow information integration across the 12-lead ECG data, the number of input channels increases gradually for each convolution layer, with a max pooling layer between each convolution layer. For example, in one implementation, following the initial atrous convolution layer, the model proceeds along the following path: one-channel convolution layer→two-channel convolution layer→max pooling layer→three-channel convolution layer→max pooling layer→four-channel convolution layer→1D convolution→average pooling layer→fully connected layer. The output of the model 300 is an indication of the risk of SCA of the subject, also referred to as the DL-ECG.

To train the model 300, a training dataset can be formed from received electrocardiograph (ECG) data. The training dataset can include an active portion and a control portion. The active portion includes a first plurality of sets of ECG data, where each set is generated from a patient that experienced a subsequent cardiac arrest (e.g., after the ECG data was generated). The control portion includes a second plurality of sets of ECG data, where each set is generated from a control subject that did not experience a subsequent cardiac arrest. The machine learning model 300 can then be trained using the training dataset (e.g., the sets of ECG data and the outcome of subsequent cardiac arrest or no subsequent cardiac arrest for each set of ECG data). In some implementations, training the machine learning model is done with a learning rate of 1×10³, a batch size of 500, an epoch count of 55, or any combination thereof.

In some implementations, each set of ECG data includes data from a 12-lead ECG. In some implementations, each set of ECG data includes ECG data spanning an identical time period, such as 2.5 seconds. In some implementations, forming the training dataset includes restricting the ECG data to a specified period of time, such as 2.5 seconds. In some implementations, forming the training dataset includes discarding any set of ECG data indicating the presence of atrial fibrillation, atrial flutter, a paced rhythm, or any combination thereof. In some cases, for of the first plurality of sets of ECG data in the active portion, the average time between the generation of the ECG data and the subsequent cardiac arrest is between 1.5 years and 2.5 years. In some implementations, a set of ECG data is considered to be from a control subject is no cardiac arrest was experienced within a threshold period of time after the generation of the ECG data, such as 1.5 years, 2, years, 2.5 years, 3 years, or any other suitable period of time.

Disclosed herein is an example of the method 200 illustrated in FIG. 2 and the DL model 300 illustrated in FIG. 3.

### Methods

### Study Design

In this example, two geographically separate community-based, prospective, and ongoing studies of out-of-hospital SCAs (e.g., subjects determined to have suffered from sudden cardiac arrest) in the general population were used. The subjects in the first study were used for training, validation, and testing, and the subjects in the second study were used for external validation. Given that coronary artery disease (CAD) is the most common underlying substrate for SCA, the control group was designed to represent a control sample with a similar prevalence of previously diagnosed CAD.

### SCA Cohorts

The first study (used for training, validation, and internal testing) ascertained all out-of-hospital SCAs from the Portland, Oregon metro area (population ~1 million), and the second study (used for external validation) ascertained all out-of-hospital SCAs from Ventura County, California (population ~850,000), each using an identical approach. Potential SCA cases in the community were identified in collaboration with each region's emergency medical services (EMS) system. Subsequently, established adjudication methods to confirm likely cardiac etiology of SCA were employed by trained physician-researchers; using all available medical record data for each potential SCA case, EMS prehospital care reports, medical examiner's reports, and death certificates from Oregon and California state vital statistics records. SCA was defined as a sudden loss of pulse due to a likely cardiac etiology that occurred with a rapid witnessed collapse, or if unwitnessed, the subject should have been seen alive within 24 hours. Successfully resuscitated cases were included in addition to non-survivors. Cases of likely non-cardiac etiology (e.g., trauma or substance abuse) or chronic terminal illness were excluded.

All cases with archived resting 12-lead ECGs available for analysis were included. These ECGs were recorded prior to and unrelated to the SCA event, with a calibration of 10 mm/mV and paper speed of 25 mm/s. ECGs with paced rhythm, atrial fibrillation, or atrial flutter were excluded a priori to create a DL model that could be applied to ECGs in sinus rhythm. Pre-arrest clinical records and ECGs were available if the patient provided written consent or was deceased, in which case consent was waived.

### Control Population

Control subjects were recruited from the Portland Oregon metro area to represent corresponding patients from the general population. Control subjects were identified through multiple sources, including patients undergoing angiography, patients having their chest pain assessed by EMS, or patients visiting an outpatient cardiology clinic. The control subjects were ascertained so that the prevalence of CAD and MI was comparable to SCA cases. Control patients had no previous history of cardiac arrest or ventricular arrhythmias. Matching cases and controls for underlying CAD enables the development of a DL model that identifies high-risk patients from a clinically comparable ‘intermediate-risk’ group. ECGs were obtained and archived in an identical manner to SCA cases.

In both SCA cases and controls, paper 12-lead ECG recordings were scanned, and digitized using software (ECGScan), which has been demonstrated to provide a robust reconstruction of a digital ECG waveform. Due to the variable length of ECG leads, the length of each lead in each sample was restricted to a 2.5-second strip, which was the minimum length of ECG waveform for each lead. Hence, digital 2.5-second strips of each lead in the 12-lead ECG were used as input for the DL model.

### Cohorts

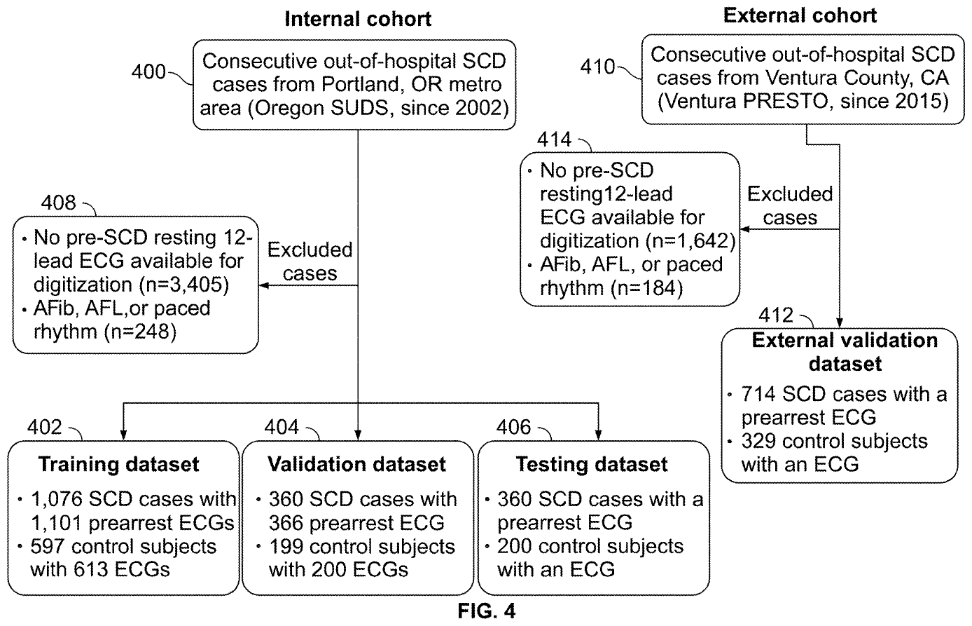

FIG. 4 shows the internal cohort 400 from the first study. The internal cohort 400 includes an internal training dataset 402, an internal validation dataset 404, and an internal testing dataset 406, and a group of excluded cases 408. The internal training dataset 402 includes 1,076 SCA cases with 1,101 prearrest ECGs, and 597 control subjects with 613 ECGs. The internal validation dataset 404 includes 306 SCA cases with 366 prearrest ECGs, and 199 control subjects with 200 ECGs. The internal testing dataset 406 includes 360 SCA cases that each include a prearrest ECG, and 200 control subjects that each include a prearrest ECG. The group of excluded cases 408 from the internal cohort 400 includes 3,405 SCA cases where no pre-SCD resting 12-lead ECG was available for digitization, and 248 cases where the ECG indicated atrial fibrillation, atrial flutter, or a paced rhythm. FIG. 4 also shows the external cohort 410 from the second study, which includes an external validation dataset 412 and a group of excluded cases 414. The external validation dataset 412 includes 714 SCA cases that each include a prearrest ECG, and 329 control subjects that each include an ECG. The group of excluded cases 414 from the external cohort 410 includes 1,642 SCA cases where no pre-SCD resting 12-lead ECG was available for digitization, and 184 cases where the ECG indicated atrial fibrillation, atrial flutter, or a paced rhythm.

### Deep Learning Model Development and Training

The DL model 300 shown in FIG. 3 (e.g., the convolutional neural network) for ECG interpretation was developed. The DL model 300 was trained on the internal training dataset 402, which included 1,101 prearrest 12-lead ECGs from 1,076 SCA, and 613 12-lead ECGs from 597 control subjects. The internal validation dataset 404 of 366 prearrest ECGs from 360 SCA cases and 200 control ECGs from 199 control subjects was used to determine when to stop model training. The ECGs were divided at the patient level so that multiple ECGs from the same patient were included in the same cohort. In the internal training dataset 402 and the internal validation dataset 404, multiple ECGs were used per patient, but in the internal testing dataset 406 and the external validation dataset 412, only one ECG was used per patient (the closest ECG that was unrelated to the SCA event). The mean time from ECG to SCA was 2.0±2.7 years in the internal cohort 400 and 1.6±2.1 years in the external cohort 410. The DL model 300 was trained using the PyTorch DL framework, and the Adam optimizer with default parameters (initial learning rate of 1×10³) with a batch size of 500 and for 55 epochs. Based on the area under the curve (AUC) of the receiver operating characteristic (ROC) curve in the internal validation dataset 404, early stopping was performed for training. The output of the DL model 300 is an indication of the risk of SCA of the subject, also referred to as the DL-ECG.

### Statistical Analyses

All continuous variables are expressed as mean±standard deviation. After model development and training, statistical analyses were performed on the internal testing dataset 406 and the external validation dataset 412 which were never seen during model training. The DL model 300's performance in identifying SCA cases was calculated by the AUC of the ROC. The DL model 300 was compared to a previously developed conventional ECG electronic risk score, which evaluates the sum of 6 ECG risk markers: resting heart rate >75 bpm, LVH, delayed QRS transition, QRS-T angle >90°, prolonged QTc, and prolonged Tpeak-to-Tend interval. Logistic regression was performed on the internal testing dataset 406 and the external validation dataset 412 using clinical variables (age, sex, heart failure, coronary artery disease, myocardial infarction, diabetes, chronic obstructive pulmonary disease, seizure, and cerebrovascular accident) with and without DL-ECG index. The best threshold for the DL model 300 was selected by maximizing the F1 metric (the harmonic mean of the precision and recall, expressed as

$$2 \times \frac{precision \times recall}{precision + recal} or \frac{2 tp}{2 tp + fp + fn} )$$

on the external validation dataset 412, and this threshold was used to report sensitivity and specificity on the test sets. Similarly, the threshold to report sensitivity and specificity for the conventional ECG electronic risk score and logistic regression models was also selected by maximizing the F1 metric. For each calculation, two-sided 95% confidence intervals (CI) were computed by bootstrapping randomly sampled 50% of the test set for 1,000 iterations. Statistical analyses were performed using Python and R.

### Results

### Demographic and Clinical Findings

The overall sample consists of a total of 2,510 SCA cases: 1,796 SCA cases from the first study (training, validation, and testing) and 196 SCA cases from the geographically distinct second study (external validation). In comparison to the SCA cases in the first study, the SCA cases in the second study were older (72.3±14.2 years vs. 67.5±14.9 years) and more often female (41.3% vs. 35.4%). The prevalence of Hispanic ethnicity (30.7% vs. 2.4%) and Asian race (7.8% vs. 3.3%) was higher in the second study, while the prevalence of White (82.0% vs. 57.6%) and Black race (10.1% vs. 2.1%) was higher in the first study. The prevalence of diabetes was 45.4% in the first study and 53.2% in the second study. Previously diagnosed heart failure (31.1% vs. 39.8%) and history of myocardial infarction (MI) (27.5% vs. 38.4%) were lower in the second study compared to the first study, respectively. The prevalence of COPD was similar (26.6% in the first study vs. 22.3% in the second study).

In comparison to SCA cases, control subjects had a similar prevalence of previously diagnosed CAD (51.2%) and MI (30.7%). However, control subjects were slightly younger (65.4=11.6 years) and had a somewhat lower prevalence of previously diagnosed diabetes (27.8%), atrial fibrillation (13.4%), heart failure (12.8%), and COPD (9.1%). Demographics and clinical characteristics of SCA cases and control subjects are presented in Table 1.

TABLE 1

| | Internal Testing Cohort | | External Validation Cohort | |
|---|---|---|---|---|
| | SCA cases in first | Control subjects | SCA cases in second | Control subjects |
| | study (n = 1,796) | (n = 996) | study (n = 714) | (n = 329) |
| Age, years | 67.5 ± 14.9 | 65.3 ± 11.6 | 65.7 ± 11.7 | 65.7 ± 11.7 |
| Female sex | 635/1,796 (35.4%) | 327/996 (32.8%) | 98/329 (29.8%) | 98/329 (29.8%) |
| Race/ethnicity, n (%) | | | | |
| White | 1,448/1,765 (82.0%) | 836/979 (85.4%) | 411/714 (57.6%) | 273/328 (83.2%) |
| Black | 178/1,765 (10.1%) | 105/979 (10.7%) | 15/714 (2.1%) | 33/328 (10.1%) |
| Asian | 59/1,765 (3.3%) | 15/979 (1.5%) | 56/714 (7.8%) | 4/328 (1.2%) |
| Hispanic | 43/1,765 (2.4%) | 13/979 (1.3%) | 219/714 (30.7%) | 11/328 (3.4%) |
| Other | 37/1,765 (2.1%) | 10/979 (1.0%) | 13/714 (1.8%) | 7/328 (2.1%) |
| Prior Medical History | | | | |
| Coronary artery | 850/1,796 (47.3%) | 502/996 (50.4%) | 257/714 (36.0%) | 176/329 (53.5%) |
| disease, n (%) | | | | |
| Diabetes, n (%) | 815/1,795 (45.4%) | 282/984 (28.7%) | 380/714 (53.2%) | 87/328 (26.5%) |
| History of MI, n (%) | 689/1,796 (38.4%) | 302/996 (30.3%) | 196/714 (27.5%) | 105/329 (31.9%) |
| Atrial fibrillation, n (%) | 428/1,796 (23.8%) | 137/996 (13.8%) | 187/714 (26.2%) | 41/329 (12.5%) |
| CVA, n (%) | 337/1,795 (18.8%) | 83/984 (8.4%) | 120/714 (16.8%) | 18/328 (5.5%) |
| Heart failure, n (%) | 714/1,796 (39.8%) | 136/996 (13.7%) | 222/714 (31.1%) | 33/329 (10.0%) |
| COPD, n (%) | 478/1,795 (26.6%) | 89/984 (9.0%) | 159/714 (22.3%) | 31/328 (9.5%) |
| Seizure, n (%) | 150/1,795 (8.4%) | 25/984 (2.5%) | 36/714 (5.0%) | 7/328 (2.1%) |
| Syncope, n (%) | 186/1,795 (10.4%) | 50/984 (5.1%) | 78/714 (10.9%) | 11/328 (3.4%) |

### DL Model Performance

In the internal testing dataset 406, the DL model 300 achieved an AUC of 0.889 (95% CI 0.861-0.917) in detecting SCA cases from controls. Sensitivity and specificity were 0.843 (0.809-0.878) and 0.818 (0.764-0.872), respectively. In the external validation dataset 412, the DL model 300 achieved a comparable AUC of 0.820 (0.794-0.847) in detecting SCA cases. The sensitivity was 0.763 (0.733-0.796), while the specificity was 0.796 (0.753-0.838).

### Conventional ECG Electrical Risk Score Performance

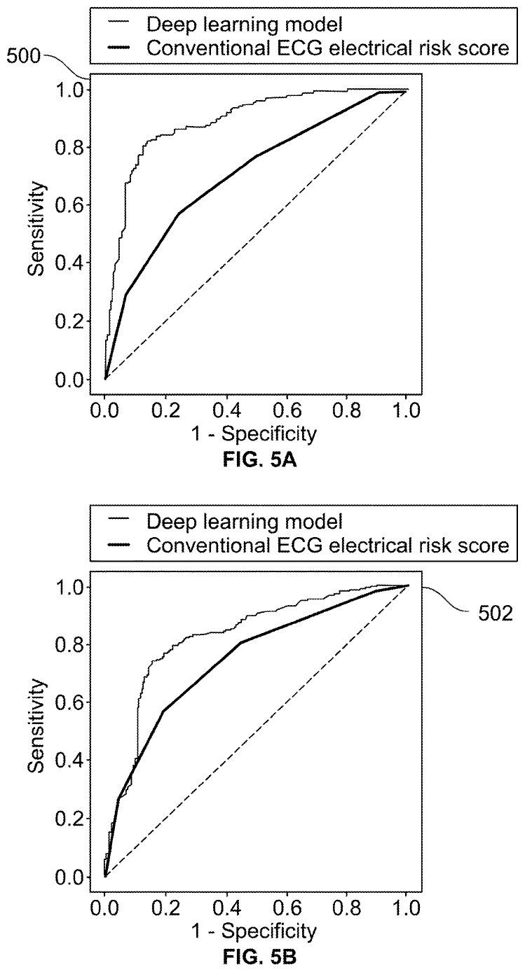

The DL model 300's performance was compared to a previously developed and validated 6-variable ECG electrical risk score that was independently associated with SCA. In the internal testing dataset 406 and the external validation dataset 412, the ECG electrical risk score achieved AUCs of 0.712 (0.668-0.756) and 0.743 (0.711-0.775) in detecting SCA cases from controls, respectively. The sensitivity was 0.779 (0.721-0.837) in the internal testing dataset 406 and 0.569 (0.515-0.623) in the external validation dataset 412. The specificity was 0.506 (0.454-0.558) in the internal testing dataset 406 and 0.802 (0.773-0.832) in the external validation dataset 412. Performance metrics in the internal testing dataset 406 and the external validation dataset 412 for the DL model 300 and the conventional ECG electrical risk score are presented in Table 2. An AUC curve 500 for the internal testing dataset 406 vs. the conventional ECG electrical risk score is shown in FIG. 5A, and an AUC curve 502 for the external validation dataset 412 vs. the conventional ECG electrical risk score is shown in FIG. 5B.

TABLE 2

| | AUC (95% CI) | Sensitivity (95% CI) | Specificity (95% CI) |
|---|---|---|---|
| Testing Cohort | | | |
| DL Model | 0.889 (0.861-0.917) | 0.843 (0.809-0.878) | 0.818 (0.764-0.872) |
| Conventional ECG | 0.712 (0.668-0.756) | 0.779 (0.721-0.837) | 0.506 (0.454-0.558) |
| electrical risk score | | | |
| External Validation Cohort | | | |
| DL Model | 0.820 (0.794-0.847) | 0.763 (0.733-0.796) | 0.796 (0.753-0.838) |
| Conventional ECG | 0.743 (0.711-0.775) | 0.569 (0.515-0.623) | 0.802 (0.773-0.832) |
| electrical risk score | | | |

### Logistic Regression Models

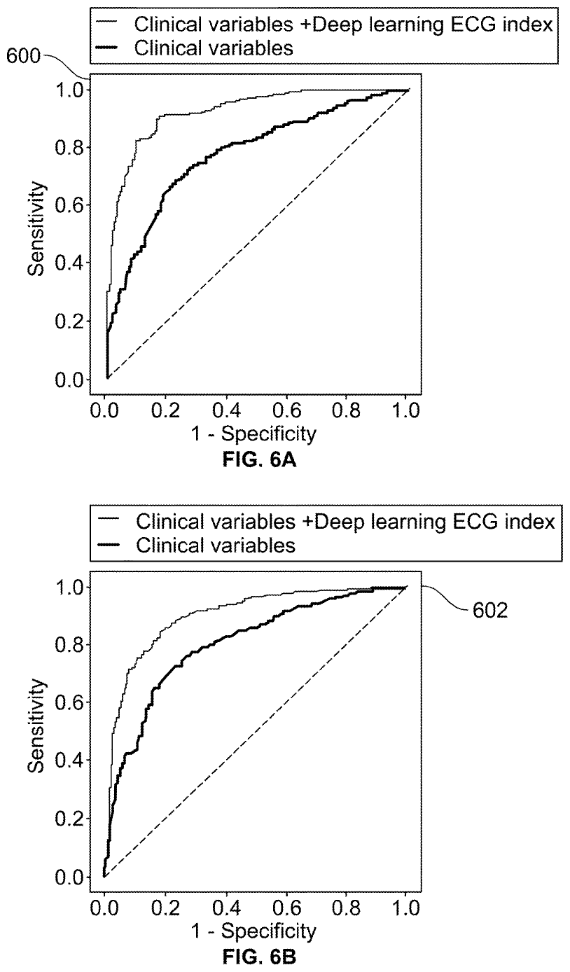

To evaluate the predictive power of DL-ECG index beyond conventional clinical SCA risk factors, logistic regression analyses were performed including clinical variables with and without DL-ECG index in the internal testing dataset 406 and the external validation dataset 412. In the internal testing dataset 406, addition of the DL-ECG index into clinical variables improved the discriminative value of SCA from an AUC of 0.780 (0.741-0.818) to an AUC of 0.919 (0.895-0.943). Similar results were obtained in the external validation dataset 412, in which addition of the DL-ECG index into clinical variables improved the discriminative value of SCA from an AUC of 0.806 (0.778-0.833) to an AUC of 0.899 (0.878-0.920). Regression model performance metrics in the internal testing dataset 406 and the external validation dataset 412 are presented in Table 3 and AUC curves in FIG. 6. An AUC curve 600 for the internal testing dataset 406 vs. the regression model is shown in FIG. 6A, and an AUC curve 602 for the external validation dataset 412 vs. the regression model is shown in FIG. 6B.

TABLE 3

| | AUC (95% CI) | Sensitivity (95% CI) | Specificity (95% CI) |
|---|---|---|---|
| Internal Testing Cohort | | | |
| Clinical variables | 0.780 (0.741-0.818) | 0.773 (0.714-0.831) | 0.644 (0.595-0.694) |
| Clinical variables + DL- | 0.919 (0.895-0.943) | 0.763 (0.703-0.822) | 0.914 (0.885-0.943) |
| ECG index | | | |
| External Validation Cohort | | | |
| Clinical variables | 0.806 (0.778-0.833) | 0.777 (0.732-0.823) | 0.702 (0.668-0.735) |
| Clinical variables + DL- | 0.899 (0.878-0.920) | 0.808 (0.765-0.851) | 0.842 (0.815-0.869) |
| ECG index | | | |

### Discussion

Data from two large geographically distinct community-based out-of-hospital SCA cohorts was utilized to train, test, and validate a 12-lead ECG waveform-based DL model 300 that distinguishes individuals who suffered SCA from controls. The DL model 300 achieved a high accuracy with an AUC of 0.889 for the internal testing dataset 406 and 0.820 for the external validation dataset 412, suggesting that ECG-based DL models harbor significant potential for augmenting SCA risk stratification. Moreover, given the differences in demographics and clinical characteristics between SCA cases in the internal testing dataset 406 and the external validation dataset 412, these results could potentially generalize well in other populations.

There are some unique aspects of study design that made this work feasible. SCA is a dynamic and unexpected event that requires prospective ascertainment. Since annual incidence is in the range of 50-100/100,000, existing cohorts of 5000-10000 subjects cannot yield sufficient numbers of SCA cases for viable analyses, especially those that employ deep learning models. The establishment of the internal cohort 400 and the external cohort 410 consisting of approximately 1.85 million US residents, provided sufficient numbers for deep learning. Equally important, both studies have been obtaining and archiving digitized 12-lead ECGs performed prior, and unrelated to SCA events. While this is a challenging process for the SCA phenotype, it is a pre-requisite for discovery of prediction models.

The association between ECG abnormalities and increased risk of SCA has been established by multiple studies, in which several abnormalities in heart rhythm, heart rate, depolarization, and repolarization have been linked with increased risk. However, the predictive power of single ECG abnormalities is low. Combinations of ECG variables as risk scores yields better results. However, determining the optimal number of ECG variables in conventional risk stratification models is a challenge. Furthermore, these models are limited by low throughput due to the use of ECG variables that may not be measured by conventional ECG interpretation computers, and thus requiring customized measurement or involvement of specially trained experts. In contrast to conventional risk calculators, DL models do not require manual feature selection and extraction but instead can utilize the entire digital signal to incorporate novel indices of risk. ECG DL models have the potential to achieve higher throughput and broader scope while preserving accuracy. The DL model 300 disclosed herein also achieved significantly higher performance in detecting SCA cases, which supports the higher utility of DL models.

SCA is a complex trait as well as a multifactorial event, and pathophysiology is based on the interplay between the underlying substrate and a variety of triggers. ECG abnormalities that have been associated with an increased risk of SCA are often surrogates of the underlying cardiac substrate (e.g., LVH, myocardial scarring, repolarization abnormality), and accurate risk stratification requires combination of several nonspecific ECG abnormalities. Despite the fact that the ECG may reflect widespread cardiac and noncardiac conditions, the logistic regression model showed that DL-ECG index generated by the DL model 300 improved the discriminative value of SCA over clinical variables. In comparison to conventional dichotomous analytical methods, deep learning based ECG analysis may provide more precise and comprehensive quantification of ECG abnormalities and deeper phenotyping.

### Alternative Implementations

Alternative Implementation 1. A method of determining risk of cardiac arrest in a subject, the method comprising: receiving electrocardiograph (ECG) data of the subject; inputting at least a portion of the ECG data into a trained machine learning model; and receiving from the trained machine learning model an indication of the risk of cardiac arrest in the subject.

Alternative Implementation 2. The method of Alternative Implementation 1, wherein the indication of the risk of cardiac arrest includes an indication of whether the subject will experience cardiac arrest within a redetermined period of time, a probability that the subject will experience cardiac arrest within the predetermined period of time, or both.

Alternative Implementation 3. The method of Alternative Implementation 1 or Alternative Implementation 2, wherein the ECG data includes data from a 12-lead ECG, data from a 3-lead ECG, data from a 4-lead ECG, data from a 3-electrode ECG, data from a 5-electrode ECG, data from a 10-electrode ECG, data from a Holter monitor, data from a smartwatch, or any combination thereof.

Alternative Implementation 4. The method of any one of Alternative Implementations 1 to 3, wherein the portion of the ECG data is an ECG sample having a predefined duration.

Alternative Implementation 5. The method of any one of Alternative Implementations 1 to 4, further comprising extracting an ECG sample from the ECG data having a predefined duration, to thereby form the portion of the ECG data that is input into the trained machine learning model.

Alternative Implementation 6. The method of any one of Alternative Implementations 1 to 5, wherein the ECG data is 12-lead ECG data, and wherein the method further comprises extracting, from each lead of the 12-lead ECG data, a sample having a predefined duration.

Alternative Implementation 7. The method of Alternative Implementation 6, wherein inputting at least the portion of the ECG data into the trained machine learning model includes inputting the sample of each lead of the 12-lead ECG data into the trained machine learning model.

Alternative Implementation 8. The method of Alternative Implementation 6 or Alternative Implementation 7, wherein each sample of the 12-lead ECG has an identical predefined duration.

Alternative Implementation 9. The method of Alternative Implementation 6 or Alternative Implementation 7, wherein at least two samples of the 12-lead ECG have different predefined durations.

Alternative Implementation 10. The method of any one of Alternative Implementations 4 to 9, wherein the predefined duration is less than or equal to 10 seconds.

Alternative Implementation 11. The method of any one of Alternative Implementations 4 to 10, wherein the predefined duration is less than or equal to 5 seconds.

Alternative Implementation 12. The method of any one of Alternative Implementations 4 to 11, wherein the predefined duration is less than or equal to 2.5 seconds.

Alternative Implementation 13. A method of training a machine learning model to determine a risk of cardiac arrest in a subject, the method comprising: receiving electrocardiograph (ECG) data; forming a training dataset from the ECG data, the training dataset including an active portion and a control portion, wherein the active portion includes a first plurality of sets of ECG data, each generated from a patient that experienced a subsequent cardiac arrest, and the control portion includes a second plurality of sets of ECG data, each generated from a control subject that did not experience a subsequent cardiac arrest; and training the machine learning model using the training dataset.

Alternative Implementation 14. The method of Alternative Implementation 13, wherein each set of ECG data in the first plurality of sets of ECG data and the second plurality of sets of ECG data includes data from a 12-lead ECG.

Alternative Implementation 15. The method of Alternative Implementation 13 or Alternative Implementation 14, wherein each set of ECG data in the first plurality of sets of ECG data and the second plurality of sets of ECG data includes ECG data over an identical time period.

Alternative Implementation 16. The method of Alternative Implementation 15, wherein the identical time period is 2.5 seconds.

Alternative Implementation 17. The method of Alternative Implementation 13, wherein forming the training dataset includes restricting each set of ECG data in the first plurality of sets of ECG data and the second plurality of sets of ECG data to an identical time period.

Alternative Implementation 18. The method of Alternative Implementation 17, wherein the identical time period is 2.5 seconds.

Alternative Implementation 19. The method of Alternative Implementation 13, wherein forming the training data set includes discarding sets of ECG data indicating a presence of atrial fibrillation, atrial flutter, a paced rhythm, or any combination thereof.

Alternative Implementation 20. The method of Alternative Implementation 13, wherein an average time between (i) a generation of each set of ECG data in the first plurality of sets of ECG data and (ii) the subsequent cardiac arrest, is between 1.5 years and 2.5 years.

Alternative Implementation 21. The method of Alternative Implementation 13, wherein training the machine learning model includes training the machine learning model with a learning rate of 1×10³, a batch size of 500, and an epoch count of 55.

Alternative Implementation 22. A system comprising a control system configured to implement the method of any one of Alternative Implementations 1 to 21.

Alternative Implementation 23. A computer program product comprising instructions which, when executed by a computer, cause the computer to carry out the method of any one of Alternative Implementations 1 to 21.

Alternative Implementation 25. The computer program product of Alternative Implementation 23, wherein the computer program product is a non-transitory computer readable medium.

Alternative Implementation 26. A system comprising: a memory device having stored thereon machine-readable instructions; and a control system including one or more processors configured to execute the machine-readable instructions to: receive electrocardiograph (ECG) data of a subject; input at least a portion of the ECG data into a trained machine learning model; and receive from the trained machine learning model an indication of a risk of cardiac arrest in the subject.

Alternative Implementation 27. A system comprising: a memory device having stored thereon machine-readable instructions; and a control system including one or more processors configured to execute the machine-readable instructions to: receive electrocardiograph (ECG data; form a training dataset from the ECG data, the training dataset including an active portion and a control portion, wherein the active portion includes a first plurality of sets of ECG data, each generated from a patient that experienced a subsequent cardiac arrest, and the control portion includes a second plurality of sets of ECG data, each generated from a control subject that did not experience a subsequent cardiac arrest; and train the machine learning model using the training dataset.

One or more elements or aspects or steps, or any portion(s) thereof, from one or more of any of the claims or Alternative Implementations can be combined with one or more elements or aspects or steps, or any portion(s) thereof, from one or more of any of the other claims or Alternative Implementations or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method of determining risk of cardiac arrest in a subject, the method comprising:
receiving electrocardiograph (ECG) data of the subject;
inputting at least a portion of the ECG data into a trained machine learning model; and
receiving from the trained machine learning model an indication of the risk of cardiac arrest in the subject.

2. The method of claim 1, wherein the indication of the risk of cardiac arrest includes an indication of whether the subject will experience cardiac arrest within a predetermined period of time, a probability that the subject will experience cardiac arrest within the predetermined period of time, or both.

3. The method of claim 1, wherein the ECG data includes data from a 12-lead ECG, data from a 3-lead ECG, data from a 4-lead ECG, data from a 3-electrode ECG, data from a 5-elecrode ECG, data from a 10-electrode ECG, data from a Holter monitor, data from a smartwatch, or any combination thereof.

4. The method of claim 1, wherein the portion of the ECG data is an ECG sample having a predefined duration.

5. The method of claim 1, further comprising extracting an ECG sample from the ECG data having a predefined duration, to thereby form the portion of the ECG data that is input into the trained machine learning model.

6. The method of claim 1, wherein the ECG data is 12-lead ECG data, and wherein the method further comprises extracting, from each lead of the 12-lead ECG data, a sample having a predefined duration.

7. The method of claim 6, wherein inputting at least the portion of the ECG data into the trained machine learning model includes inputting the sample of each lead of the 12-lead ECG data into the trained machine learning model.

8. The method of claim 6, wherein each sample of the 12-lead ECG has an identical predefined duration.

9. The method of claim 6, wherein at least two samples of the 12-lead ECG have different predefined durations.

10. The method of claim 4, wherein the predefined duration is less than or equal to 10 seconds.

11. The method of claim 4, wherein the predefined duration is less than or equal to 5 seconds.

12. The method of claim 4, wherein the predefined duration is less than or equal to 2.5 seconds.

13. A method of training a machine learning model to determine a risk of cardiac arrest in a subject, the method comprising:
receiving electrocardiograph (ECG) data;
forming a training dataset from the ECG data, the training dataset including an active portion and a control portion, wherein
the active portion includes a first plurality of sets of ECG data, each generated from a patient that experienced a subsequent cardiac arrest, and
the control portion includes a second plurality of sets of ECG data, each generated from a control subject that did not experience a subsequent cardiac arrest; and
training the machine learning model using the training dataset.

14. The method of claim 13, wherein each set of ECG data in the first plurality of sets of ECG data and the second plurality of sets of ECG data includes data from a 12-lead ECG.

15. The method of claim 13, wherein each set of ECG data in the first plurality of sets of ECG data and the second plurality of sets of ECG data includes ECG data over an identical time period.

16. The method of claim 15, wherein the identical time period is 2.5 seconds.

17. The method of claim 13, wherein forming the training dataset includes restricting each set of ECG data in the first plurality of sets of ECG data and the second plurality of sets of ECG data to an identical time period.

18. The method of claim 17, wherein the identical time period is 2.5 seconds.

19. The method of claim 13, wherein forming the training data set includes discarding sets of ECG data indicating a presence of atrial fibrillation, atrial flutter, a paced rhythm, or any combination thereof.

20. The method of claim 13, wherein an average time between (i) a generation of each set of ECG data in the first plurality of sets of ECG data and (ii) the subsequent cardiac arrest, is between 1.5 years and 2.5 years.

21-27. (canceled)
